(19) United States
(12) Patent Application Publication
(10) Pub. No.: US 2009/0048543 A1
(43) Pub. Date: Feb. 19, 2009

(54) Method, system and module for determining the stimulated point of human body

(75) Inventor: Shan-Chi Ko, Taipei City (TW)

Correspondence Address: BACON & THOMAS, PLLC, 625 SLATERS LANE, FOURTH FLOOR, ALEXANDRIA, VA, 22314-1176, US

(73) Assignee: Shan-Chi Ko, Taipei City (TW)

(21) Appl. No.: 12/222,402

(22) Filed: Aug. 8, 2008

Related U.S. Application Data
(63) Continuation-in-part of application No. 11/889,134, filed on Aug. 9, 2007.

(30) Foreign Application Priority Data
Nov. 2, 2007 (TW) 096141554

(51) Int. Cl. A61H 1/00; A61B 5/00

(52) U.S. Cl. 601/1; 600/301

(57) ABSTRACT

The present invention discloses a method, system and module for determining the stimulated point of human body. The method includes defining the distributions of the plurality of collateral meridians of human and the plurality of body flow lines, thereby constructing the related information regarding the collateral meridians and the body flow lines based on the distributions of the plurality of collateral meridians and body flow lines, identifying the corresponding body flow lines based on the collateral meridians of the abnormal portions, determining one or a plurality of stimulated points based on the body flow fines and the corresponding information on abnormal positions, and applying the stimulations to the stimulated points for attenuating or recovering the abnormal portion. The system includes a process system to analyze the data inputted by the input/output module and data enclosed in the databases through the calculating module, and then the plurality of stimulated points can be obtained. Further, the module includes the distributions of the plurality of body flow lines and the stimulated points to determine one or a plurality of the stimulated points.

### CROSS-REFERENCE

The present application is a continuation-in-part (CIP) application of a pending U.S. patent application Ser. No. 11/889,134, entitled “Method for Determining the Stimulated Point of Human Body,” and filed on Aug. 9, 2007, which is incorporated herein by reference in its entirety.

### BACKGROUND OF THE INVENTION

1. Field of the Invention

This invention relates to a method, system and module for determining the stimulated point, and more particularly to a method, system and module for determining the collateral meridian stimulated point of human body, combining the theories of Chinese medicine and modern medicine and seasonal factors. The stimulated points are determined according to the collateral meridian stimulated point of human body, and then the stimulations are applied to attenuate or recover the abnormal portions.

2. Description of the Prior Art

Recently, medical science and technology have developed, and individuals live at a frantic pace with hectic lifestyles. Most individuals spend a lot of time on work, but have little time left over for exercise or travel. However, a few people still manage to maintain good health. Discomfort or pain is generally unavoidable with increasing age. People lack sufficient time to treat or attenuate the pain syndrome resulting in a situation where recovery becomes impossible.

Previously, people faced risks from numerous diseases. During the last several thousand years, people have been devoted to solving the problems of people's birth, age, illness and death in an effort to fight various diseases. For example, headache, shoulder and neck pain, toothache, backache and knee pain are included in the pain syndromes. Despite science and technology rapidly changing, pain remains unavoidable. Because human life generally begins with labor pains left by the mother and ends with various diseases, people must face and overcome physiological or psychological pains throughout their lives. Although some people have good resistance to pain, most people do a poor job.

Generally, when experiencing discomfort or pain, people may seek modern medical treatment because medicine or injections can be used to treat pain directly. However, treatments can provide a temporary rather than a permanent solution to attenuating the pain. Some people may undergo the Chinese medical treatment, but their symptoms are likely to take a long time to improve in this case. Treatment methods and times differ markedly between Chinese and modern medicine, and it is different to select one from them. Furthermore, the pain syndrome is a form of abnormal portion in human body. It is urgently necessary to attenuate, improve or eliminate the pain quickly and effectively.

Moreover, in modern medicine, certain medicines, such as an anodyne or an anaesthetic, are always used in treatment, and their use may be accompanied by unexpected side effects, complications or sequelas. Similarly, in Chinese medicine, the acupressure and massage may attenuate the muscle pain but cannot be employed to treat the acute pain syndrome. However, it is worse to provide the treatment of the acupressure or the massage directly on the pain portion with inflammation. To avoid the increased inflammation, people should carefully select the most appropriate treatment. People may be advised reconsider the acceptable treatments for different types and portions of pain syndrome.

Based on the above, the present invention provides a new method, system and module for determining the stimulated point on the human body to overcome the above disadvantages of prior art. The method, system and module of the present invention is developed by amending and combining the Chinese and modern medicine theory to attenuate or recover the abnormal portions, and can be modified according to different requirements.

### SUMMARY OF THE INVENTION

The present invention illustrates preferred embodiments as follows. However, it is appreciated that the present invention can extensively perform in other embodiments except for these detailed descriptions. The scope of the present invention is not limited to these embodiments and should be accorded the following claims.

One objective of the present invention is to provide a method, system and module for determining the stimulated point of human body, which provides a new aspect of five dimensions to replace with two dimensions method.

Another objective of the present invention is to provide a method, system and module for determining the stimulated point of human body, where the stimulations can be applied according to the user's requirements.

Still another objective of the present invention is to provide a method, system and module for determining the stimulated point of human body, which can offer the appropriate stimulation to attenuate or recover the abnormal portion rather than perform the treatment directly on the pain portion.

Yet another objective of the present invention is to provide a method, system and module for determining the stimulated point of human body, which is based on the combination of amended theories of the Ying-Yang and the five element, 12 collateral meridians, the relationship of inter promotion and inter-restrict of Chinese medicine and pathologic physiology of modern medicine.

Yet another objective of the present invention is to provide a method, system and module for determining the stimulated point of human body, which can offer a module with the stimulated points of human body distributed thereon and the module can be employed to determine one or a plurality of stimulated point of human body.

Another objective of the present invention is to provide a method, system and module for determining the stimulated point of human body, which the information regarding the stimulated point and the corresponding stimulation types can be obtained to attenuate or recover the abnormal portions effectively.

The present invention provides a method for determining the stimulated point of human body comprising defining the distributions of a plurality of collateral meridians in human and a plurality of body flow lines; constructing the related information regarding the collateral meridians and the body flow lines based on the distributions of the plurality of collateral meridians and the body flow lines; identifying the corresponding body flow lines based on the collateral meridians of abnormal portions; determining one or a plurality of stimulated points based on the body flow lines and the corresponding information of abnormal positions; and applying the stimulations to the stimulated points for attenuating or recovering the abnormal portions.

The present invention also provides a system for determining stimulated points of human body, comprising an input/output module to input/output data; a process system coupled to the input/output module; a data base coupled to the process system; and a calculating module coupled to said process system, wherein the process system analyzes the data obtained from the input/output module and the data base, and the input/output module can output the corresponding data of one or a plurality of stimulated points of human body.

The present invention further provides a module for determining stimulated points of human body, comprising a plurality of body flow lines configured on the module; and a plurality of stimulated points configured respectively on each of body flow lines, wherein the plurality of body flow lines and the plurality of stimulated points are defined by the theories of the amended Ying-Yang and the six elements and collateral meridians, and the abnormal portion can be attenuated or recovered after applying stimulations.

### BRIEF DESCRIPTION OF THE DRAWINGS

The foregoing aspects and many of the attendant advantages of this invention will become more readily appreciated as the same becomes better understood by reference to the following detailed description, taken in conjunction with the accompanying drawings, wherein:

FIG. 1 illustrates a flow chart of a method for determining the stimulated point of human body according to the present invention;

FIG. 2A illustrates a diagram of the body flow lines of human body that partially blocked according to the present invention;

FIG. 2B illustrates a diagram of the body flow lines of human body that completely blocked according to the present invention;

FIG. 3 illustrates a diagram of a theory of Ying-Yang and six elements which is amended based on Ying-Yang and the five elements of Chinese medicine according to the present invention;

FIG. 4 illustrates a diagram of a hexagonal relation for determining the stimulated point of human body according to the present invention;

FIG. 5 illustrates a diagram of a seasonal relation for determining the stimulated point of human body according to the present invention;

FIG. 6A illustrates diagrams of the external body flow lines of left and right hands of human body according to the present invention;

FIG. 6B illustrates diagrams of the internal body flow lines of left and right hand of human body according to the present invention;

FIG. 7A illustrates diagrams of the external body flow lines of left and right feet of human body according to the present invention;

FIG. 7B illustrates diagrams of the internal body flow lines of left and right feet of human body according to the present invention;

FIG. 8A illustrates a diagram of the visual analogue scale (VAS) scores over time for pre-stimulation, post-stimulation and pills treatment according to the second embodiment of the present invention;

FIG. 8B illustrates a diagram of the three-phase bone scan before the stimulation according to the second embodiment of the present invention;

FIG. 8C illustrates a diagram of the three-phase bone scan after the stimulation according to the second embodiment of the present invention;

FIG. 9 illustrates a diagram of a system for determining the stimulated point of human body according to the present invention;

FIG. 10 illustrates a flow chart of a system for determining the stimulated point of human body according to the present invention; and

FIG. 11 illustrates a diagram of a module for determining the stimulated point of human body according to the present invention.

### DESCRIPTION OF THE PREFERRED EMBODIMENT

In the following description, numerous specific details are provided in order to give a through understanding of embodiments of the invention. Referring now to the following description wherein the description is for the purpose of illustrating the preferred embodiments of the present invention only, and not for the purpose of limiting the same. One skilled in the relevant art will recognize, however, that the invention may be practiced without one or more of the specific details, or with other methods, components, materials, etc.

Referring now to the drawings and the following description wherein the showings and description are for the purpose of illustrating the preferred embodiments of the present invention only, and not for the purpose of limiting the same. Then, the drawings of different elements are not shown to scale. Some dimensions of the related elements are exaggerated and meaningless portions are not drawn to provide a more clear description and comprehension of the present invention.

The present invention discloses a method for determining the collateral meridian stimulated point on the human body. The proposed method is based on the theories of the Ying-Yang and the five elements, as well as 12 collateral meridians, and the relationship between inter promotion and inter-restriction of Chinese medicine and the pathological physiology of modern medicine. The present invention provides a five dimensional method for determining the related corresponding body flow lines, as follows. Notably, the body flow lines in the present invention differ from the collateral meridians mentioned by the Chinese medicine. Therefore, one of the characteristic of the present invention is the body flow lines on the human body and their definitions. The body flow lines include mobilizing or circulating molecules, such as various ions, neurotransmitters, lymph and blood.

Based on the aspect of the present invention, the “collateral meridians” describe the division of the human body longitudinally and horizontally. The cross sections between the longitudinal and horizontal lines are called the collaterals. Most of the longitudinal lines are distributed in the human body, and therefore, the body can be divided into a plurality of collateral meridians distributed throughout the human body. Furthermore, various body flow lines can be found in various systems of internal organs in humans including circulatory system, digestive system, respiratory system, nervous system and urinary system. The body flow lines include various materials or molecules mobilizing or circulating throughout the human body. For example, the materials or molecules including blood, lymph, neurotransmitters, autacoid secreted by the endocrine gland, ions, air, and water. Corresponding relationships exist among the above systems, and the communication among them can present as the plurality of body flow lines. The relationships between collateral meridians and body flow lines thus can be corresponding or relative. Based on continuous efforts and research, the present invention is developed and defined by the Applicant. The related articles and information may refer to Ko et al., Acta Anaesthesiol Taiwanica, 44:59-60 (2006), entitled “Can we do better, in addition to the pharmacological treatment, on pain: Collateral Meridian Therapy” and Ko et al., Anesthesia & Analgesia, 103(6), entitled “Collateral Meridian Therapy dramatically attenuates pain and improves functional activity of a patient with complex regional pain syndrome”. The articles are incorporated herein for reference. The references provide related information regarding the importance, utility and reappearance of the present invention.

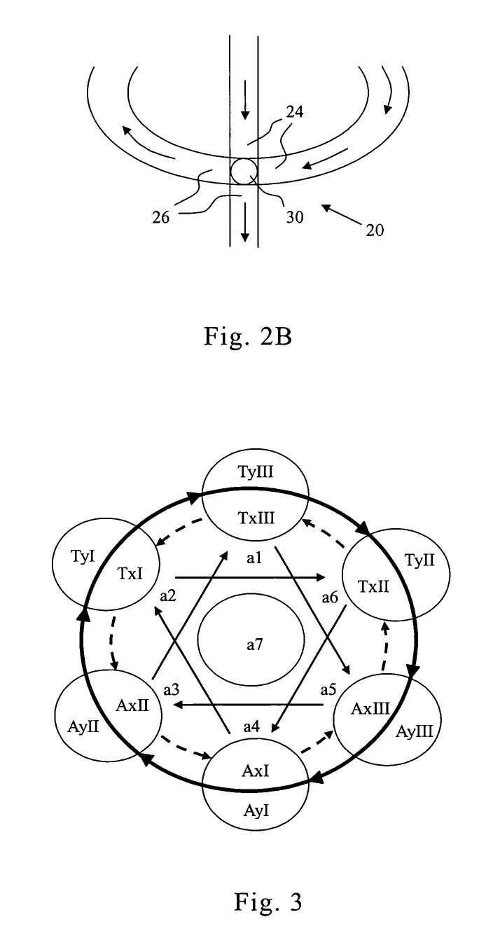

However, FIGS. 2A and 2B show that the abnormal portion of human body generated when one cross section 20 of the body flow lines is blocked by obstruction 30. According to the present invention, the type of abnormal portion includes dull pain, sharp pain or numbness, and the generation of the abnormal portion is serves as an alarm signal reflecting or indicating the abnormal physical condition. Notably, the generation of the abnormal portion is not a disease, and thus the present invention is not a diagnostic or treatment method. The above conditions are abnormal and can be attenuated or improved by the present invention.

FIG. 2A diagrammatically illustrates a cross section 20 of the body flow lines that are partially blocked by the obstruction 30 according to the present invention. When the cross section 20 is partially blocked, the mobilizing or circulating molecules cannot easily pass through body flow line channels. Individuals may then experience dull or sharp pain generated at the muscle corresponding to the previous portion 22 of the blocked section 24. FIG. 2B diagrammatically illustrates a cross section 20 of the body flow lines that completely blocked by the obstruction 30 according to the present invention. When the cross section 20 is completely blocked, the mobilizing or circulating molecules cannot pass through the body flow line channels. Individuals thus may experience the numbness generated at the muscle corresponding to the portion 26, following the blocked section 24. These pains are abnormal appearances.

Given the abnormal appearance, if the blocked cross portion 20 can communicate with the other related corresponding body flow lines and the appropriate stimulations are performed, the abnormal appearance can be attenuated or recovered. Therefore, to rapidly and effectively attenuate or recover the abnormal portion, the present invention can determine the collateral meridian stimulated points on the human body.

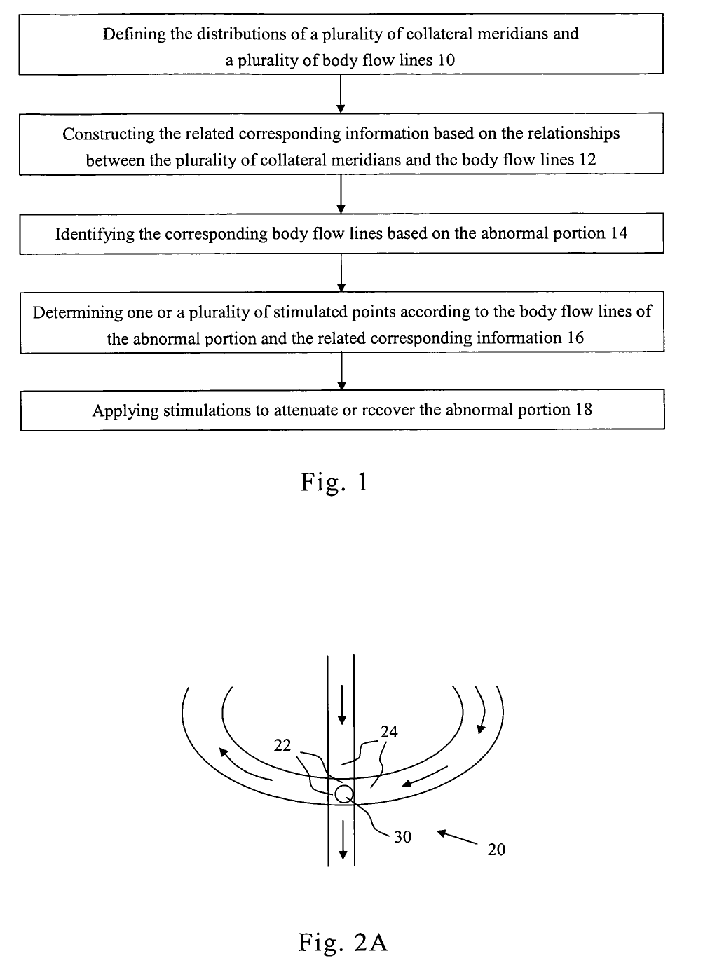

Referring to FIG. 1, it is a flow chart of a method for determining the stimulated points of the human body according to the present invention. The method comprises defining the distributions of the plurality of collateral meridians of humans and the plurality of body flow lines, as illustrated in step 10. Related information regarding the collateral medians and the body flow lines can then be constructed based on the distributions of the plurality of collateral meridians and body flow lines, as illustrated in step 12. The corresponding body flow lines can be identified according to the collateral meridians of the abnormal portions, as illustrated in step 14. Subsequently, one or a plurality of stimulated points can be determined based on the body flow fines and the corresponding information of abnormal positions, as illustrated in step 16. Specific points thus can be stimulated to achieve alleviation or recovery of the abnormal portion, as illustrated in step 18.

In one embodiment, the human collateral meridians are divided into a plurality of collateral meridians longitudinally and horizontally. One or a plurality of body flow lines are the networks that communicate with the plurality of human collateral meridians.

According to the aspect of the present invention, the human body flow lines include Ying and Yang body flow lines. The Ying body flow lines comprise the internal portions of the human body, such as the ventral region, and the internal organs of human, such as, the liver, lung, kidney, heart and spleen. Meanwhile, the Yang body flow lines comprise the external portions of human body, including the dorsal regions of human, the external portions of the left and right hands, the external portions of left and right feet, and the external organs of human, including the stomach, urchin, bladder and large and small intestine.

In one embodiment, the types of abnormal portions include chronic and acute abnormal portions, and local and central abnormal portions. For example, the central region includes the internal organs of human body, and the local region includes the limbs or trunk and the body.

In one embodiment, the abnormal portion includes the regions that are sensing certain feelings, including pain. The abnormal region can cover the head, the limbs or trunk or the body, and the visceral organs or nervous system of human body.

In a preferred embodiment, the step sequences are used to illustrate rather than limit the present invention. It is noted that the present invention can be modified and changed according to the different user's requirements.

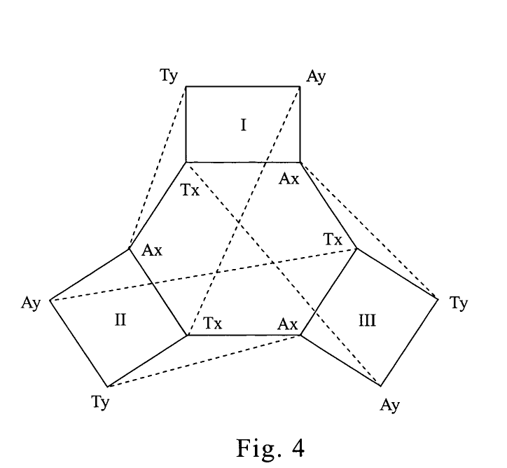

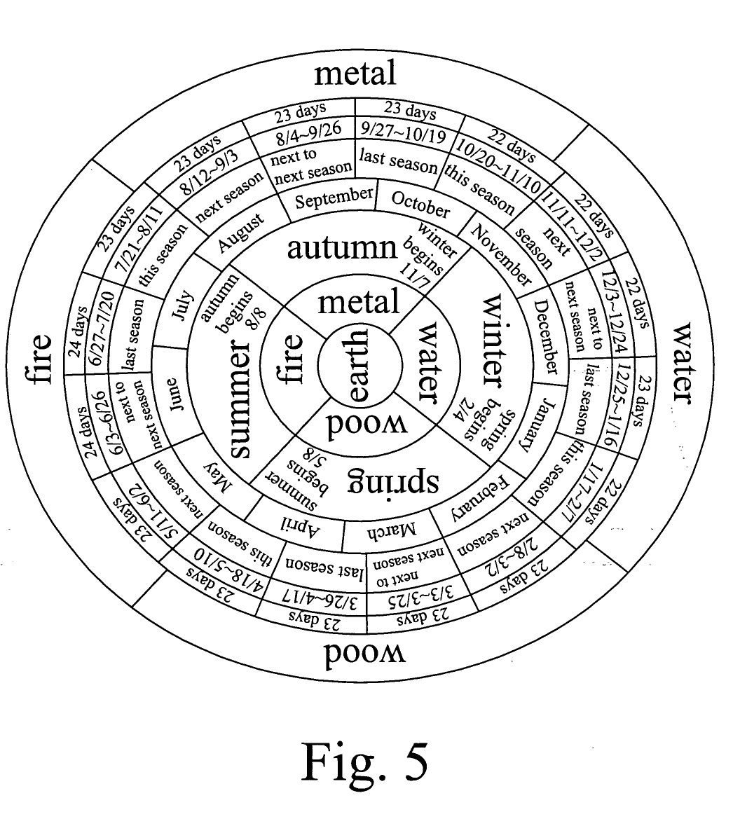

According to the aspect of the present invention, the distributions of the plurality of collateral meridians of humans and the plurality of body flow lines and one or the plurality of stimulated points are based on the corresponding or (relative) opposite relationships among the theories of the Ying-Yang and the five element shown in FIG. 3, the hexagonal relation shown in FIG. 4 and the seasonal time shown in FIG. 5.

FIG. 3 diagrammatically illustrates an amended theory of Ying-Yang and six elements based on the Ying-Yang and five elements of Chinese medicine according to the present invention. The illustrated method determines the stimulated points according to the amended theory. In FIG. 3, the references a1, a2, a3, a4, a5, a6, a7, are respectively labeled fire, metal, wood, earth, water, govern fire, air. Moreover, the references T and A are respectively represent the hands (the Japanese word for “hand” has a similar pronunciation [Te]) and feet (the Japanese word for “foot” has a similar pronunciation [Ashi]), and the references r and l respectively denote right and left. Additionally, the references x and y are respective indicate the Ying and Yang body flow lines, and each body flow line has three body flow lines, I, II and III. For examples, “TyIII” indicates that the Yang body flow lines III occupies the external portions of hands, “AxI” means that the Ying body flow lines I occupies the internal part of the foot, and “rTxI” denotes that the Ying body flow lines occupy the internal portion of the right hand.

In one embodiment, TxI denotes the lung meridian, while TxII represents the pericardium meridian, TxIII is the heart meridian, TyI denotes the large intestine meridian, TyII represents the three jiaos meridian, and TyIII is the small intestine meridian. Similarly, AxI denotes the spleen meridian, AxII represents the liver meridian, AxIII is the kidney meridian, AyI denotes to the stomach meridian, AyII represents the urchin meridian and AyIII is the bladder meridian.

Furthermore, refer to FIG. 3, the clockwise circulation relation in the outer cycle refers to the inter promotion relationship of the Ying-Yang and the five elements. For example, water generates earth and wood generates fire. The inter promotion relationship also can be termed as a mother-son relationship. The anti-clockwise circulation relation in the inner cycle is the inter-insult relation, for example that fire cannot go out when there is a lack of water. The central hexagon circulating relation is the inter-restrict relation, for example that metal can be melted by fire, wood can be pared by metal, earth can be covered by wood, and water can be absorbed by earth.

FIG. 4 shows a diagram of a hexagonal relationship for determining the stimulated point on the human body according to the present invention. The dotted lines in FIG. 4 are indicated that the communications or relationships between the inter promotion relation and the corresponding relations based on the experimental data. In the drawing, the solid lines connecting the peripheral rectangles with the central hexagon indicate the intergeneral relations among the body flow lines of Tx, Ty, Ax, Ay, I, II and III. Meanwhile, the dotted lines indicate the corresponding relationships among the body flow lines of Tx, Ty, Ax, Ay, I, II and III. For example, if the abnormal portion is generated at the body flow line TyI, the corresponding body flow line can be indicated as AxII based on the dotted line between TyI and AxII. Furthermore, if the abnormal portion is generated at the body flow line AyII, the corresponding body flow line can be indicated as TxIII based on the dotted line between AyII and TxIII.

In one embodiment, after identifying the corresponding body flow lines of the abnormal portions, the defined and related corresponding body flow lines are used to determine one or a plurality of stimulated points on the human body. Please review the following descriptions.

The distributions of body flow lines vary slightly with seasonal changes, and their properties and definitions also vary. For examples, the blood vessels expanded in summer and constrict in winter. Therefore, the present invention introduces the factors of the seasons and days to provide the proper and accurate information for determining the stimulated points of the human body for effectively attenuating or recovering the abnormal portion. FIG. 5 illustrates a diagram of a seasonal relation for determining the stimulated point of the human body according to the present invention. The drawing respectively refers to wood, fire, metal, water and earth as the seasons of spring, summer, autumn and the eighteen days before the beginning of spring, the beginning of summer, the beginning of autumn and the beginning of winter. The govern fire shown in FIG. 3 is termed the plum rains. Accordingly, the present invention provides a five-dimension method for determining the stimulated point of the human body for effectively attenuating or recovering the abnormal portion.

In one embodiment, if the blocked portion is generated at the body flow lines TxI and TyI, the pain will appear in autumn (corresponding to metal, as shown in FIG. 5), such as the Radial nerve lesion in the thumb or the forefinger. Consequently, if the suitable and accurate stimulations are offered in autumn, it may take a long time to attenuate or recover the pain portion, and the pain portion will appear frequently. If the proper stimulations are offered excluding the autumn, it is easy to attenuate or recover the pain portion.

In one embodiment, if the blocked portion is generated at the body flow lines AxIII and AyIII, the pain will appear in winter (corresponding to water, as shown in FIG. 5), with examples including lumbago and pains in the vertebra (such as the cervical vertebra, the lumbar vertebra and the caudal vertebra). Therefore, if the appropriate and accurate stimulations are offered in winter, it may take a long time to attenuate or recover the pain portion, and the pain portion will frequently appear. If the appropriate stimulations are offered excluding the winter, it may be easy to attenuate or recover the pain portion.

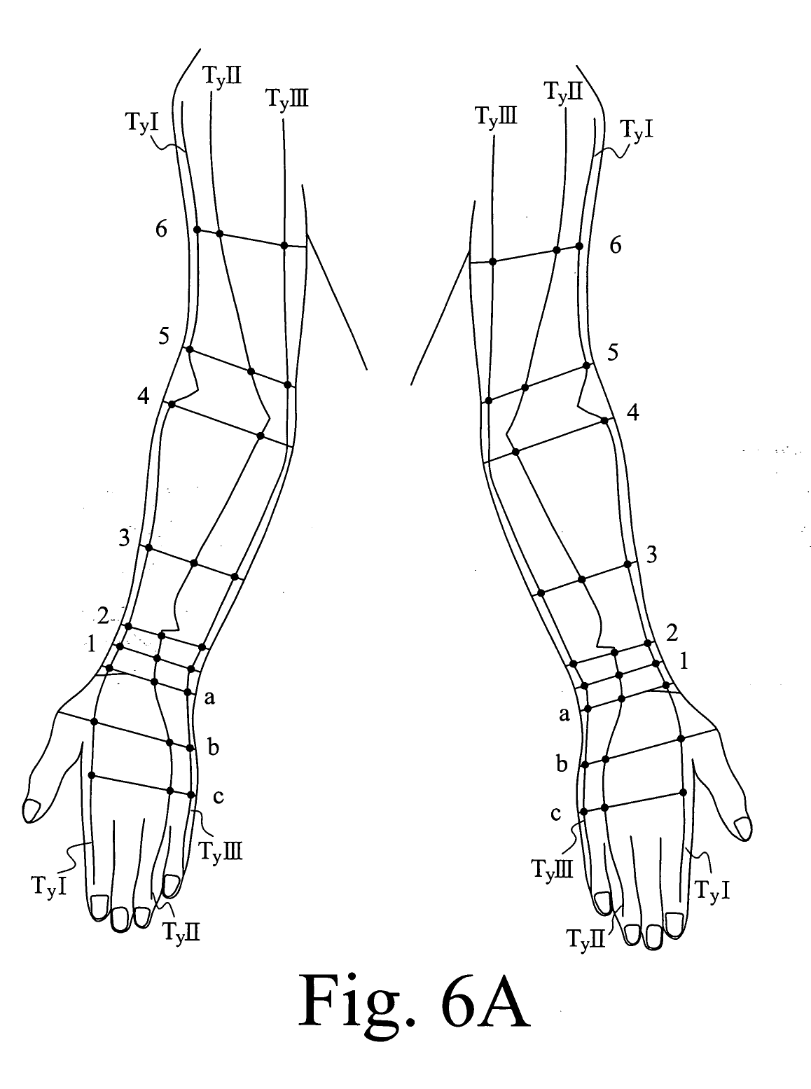

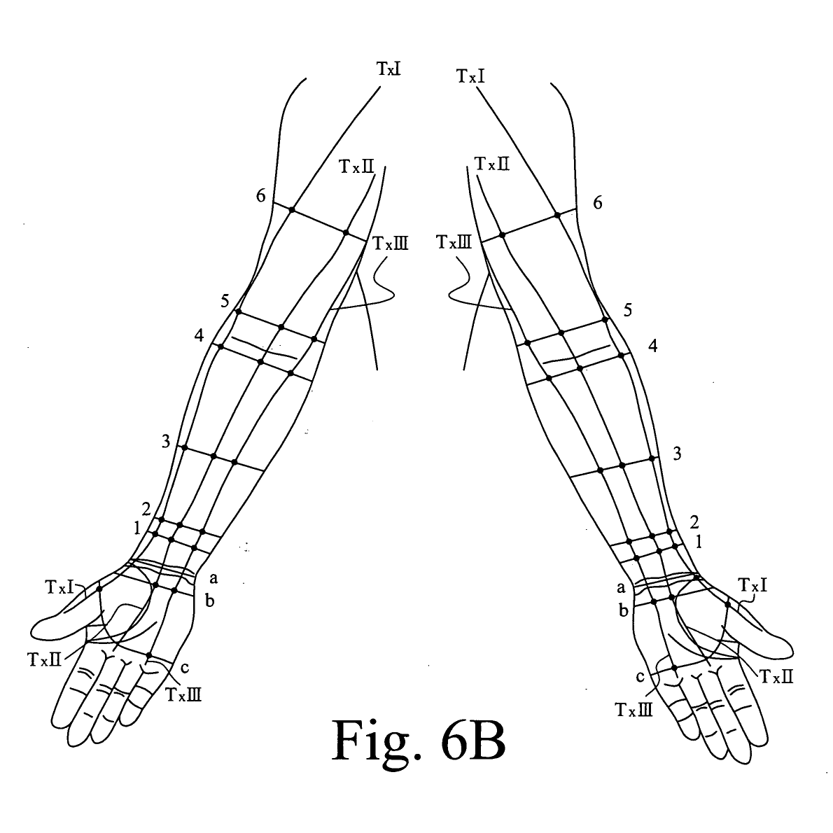

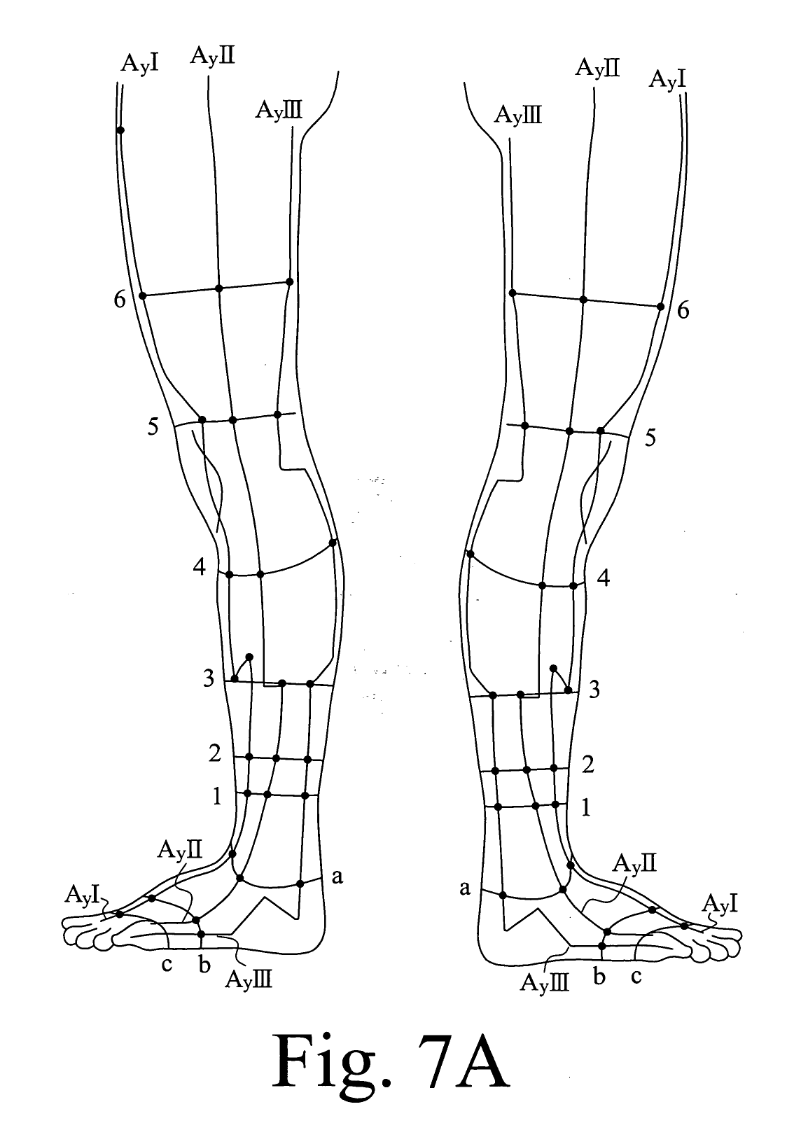

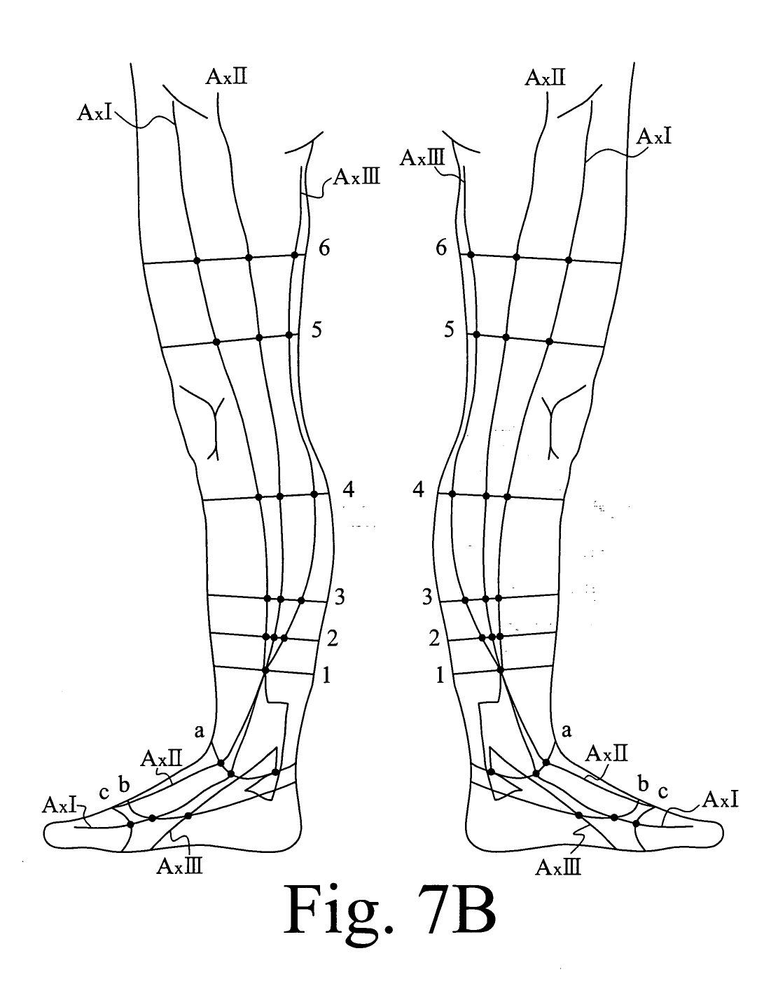

According to the aspects of the present invention, the distributions and definitions of the collateral meridian stimulated points are determined by the related information in FIGS. 6A and 6B and FIGS. 7A and 7B.

Refer to FIG. 6A, it illustrates diagrams of the external body flow lines of the left and right hands of the human body according to the present invention. FIG. 6B illustrates diagrams of the internal body flow lines of the left and right hand of human body according to the present invention. In FIGS. 6A and 6B, the internal body flow lines TxI, TxII, TxIII of the left hands and the external body flow lines TyI, TyII, TyIII of the right hands are respectively divided into a plurality of stimulated points. In the drawing, the body flow lines are divided into six stimulated points, 1, 2, 3, 4, 5, 6, at the upper elbow of the hand and three stimulated points, a, b, c, below the elbow of the hands. Accordingly, each body flow line of the hand can be divided into the nine stimulated points, a, b, c, 1, 2, 3, 4, 5, 6, as shown in FIGS. 6A and 6B.

Moreover, refer to FIG. 7A, it illustrates diagrams of the external body flow lines of the left and right feet of human body according to the present invention. FIG. 7B illustrates diagrams of the internal body flow lines of left and right feet of human body according to the present invention. In FIGS. 7A and 7B, the internal body flow lines AxI, AxII, AxIII of the left feet and the external body flow lines AyI, AyII, AyIII of the right feet are respectively divided into a plurality of stimulated points. In the drawing, the body flow lines are divided into three stimulated points, a, b, c, below the ankle and six stimulated points, 1, 2, 3, 4, 5, 6, above the ankle. Accordingly, each body flow line of the foot can be divided into the nine stimulated points, a, b, c, 1, 2, 3, 4, 5, 6, as shown in FIGS. 7A and 7B.

In one embodiment, based on the above aspects, each individual has 12 body flow lines distributed in the left and right hands and left and right feet, and therefore, resulting in the provision of 108 stimulated points according the present invention. Notably, the numbers and the positions of the body flow lines and the stimulated points are described for illustrating rather than to limit the present invention. The numbers and the positions of the body flow lines and the stimulated points can be modified according to the different conductions requirements.

In one embodiment, the stimulated points are the portions corresponding or relative to the pain portions. The relations can be obtained from the above definitions and determination method.

According to the aspect of the present invention, the plurality of stimulated points includes control stimulated points (C points) and function stimulated points (Γ points). In one embodiment, the C points indicate the stimulated points between the plurality of the stimulated points, while the F points refer to the stimulated points used to attenuate or recover the abnormal portions.

In one embodiment, the stimulations include the acupressure, massage, acupuncture, electric stimulation (ES), laser or the improved chemical or natural articles. The improved chemical or nature articles can be used externally or internally.

In one embodiment, the duration and frequency of the stimulation may be several times per days and lasting for a few minutes each time. Meanwhile, the duration and frequency of the stimulation can be modified and changed to fit user's requirements.

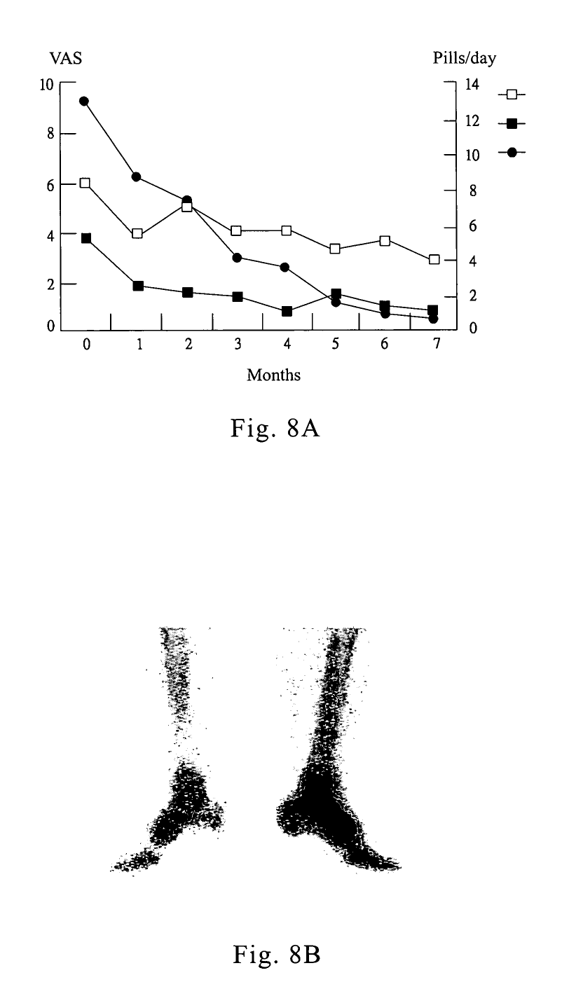

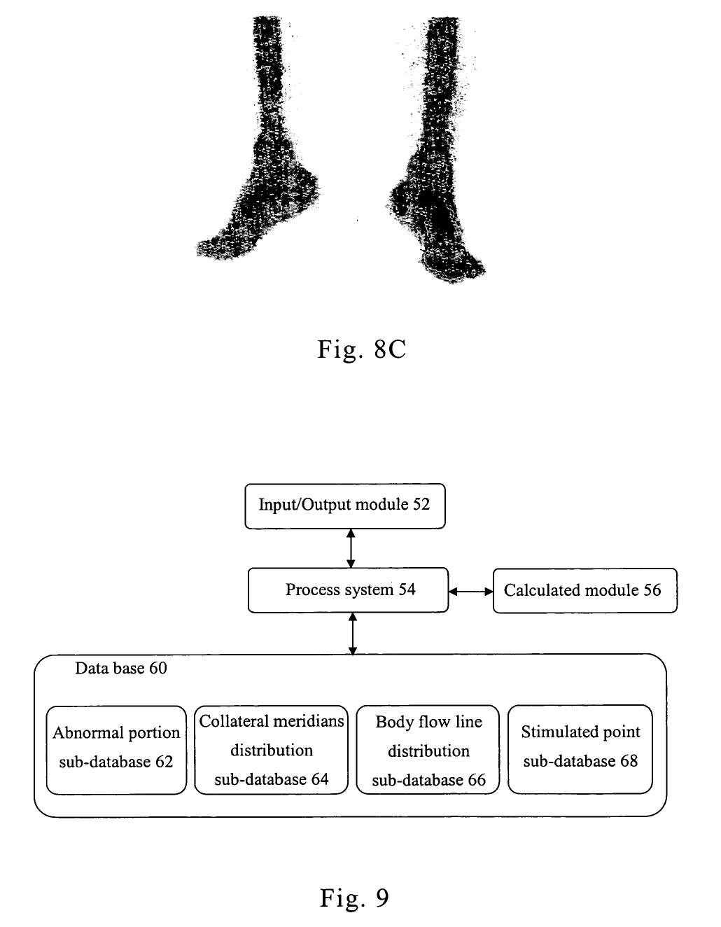

The following detailed descriptions provided the two embodiments of clinical experiments. The foregoing aspects and many of the attendant advantages of this invention will become more readily appreciated as the invention becomes better understood based on the following detailed description, taken in conjunction with the accompanying drawings, FIG. 8A, 8B, 8C. The accompanying drawings and following embodiments are used for illustrative purposes rather than to limit the present invention.

### EXAMPLE I

In Example I, a 31 year old pregnancy woman frequently suffered from the paroxysmal pain due to a combination of work and family pressures. However, the anodyne is not suitable for administration either orally or by injection during pregnancy. The paroxysmal pain cannot be solved, but the pain frequency is highly and the duration is longer with time.

According to the related information, the abnormal portion indicate the body flow line II of the external portion of the right foot (Ying meridian), rAyII. In this case, the body flow line corresponds to the internal portion of the right hand (Yang meridian), rTxIII. Based on the information regarding the distributions of collateral meridians and the body flow lines, the C point can be indicated and determined by the stimulated point 1 of the internal portion of right hand, rTxIII. Moreover, the F point can also be indicated and determined by stimulated point c. To solve the obstruction in the body flow lines stimulation thus is provided on the both stimulated points to attenuate or recover the abnormal portion. Additionally, the stimulations include acupressure, massage, acupuncture, electric stimulation (ES), laser or improved chemical or nature articles.

In Example I, the stimulation frequency may be 1-2 times and the stimulation duration may be 1-2 minutes each time. Furthermore, the stimulation frequency and duration are described to illustrate the present invention without limiting it. The present invention can be modified and changed based on the different levels of the abnormal portion and conditions.

### EXAMPLE II

In Example II, the sympathetic blockade with local anesthetics is the “gold standard” treatment for a young male with the complex regional pain syndrome (CRPS), and the visual analogue scales (VAS) scores can be termed the secondary indictors. However, the unanticipated side effects, complications or sequelas are generally followed by the conventional anesthetic method.

According to the aspect and method of the present invention, the body flow lines corresponding to the abnormal portion can be identified, and after which stimulations are provided.

Refer to FIG. 8A, it illustrates the diagram of the visual analogue scale (VAS) scores over time for pre-stimulation, post-stimulation and pill treatment, well known prior art, according to Example II of the present invention. The X-axis indicates the time (months per unit), the left Y-axis indicates the visual analogue scales (VAS) scores and the right Y-axis indicates the analgesic pills (pills/day). In the drawing, the open boxes show the VAS scores before the stimulation, while the closed boxes show the VAS scores after the stimulation and the closed circle shows the VAS scores after the pill treatment. According to the diagram, after the stimulation, the VAS scores reduce over time and are lower than the VAS scores before the stimulation and after the pill treatment. Therefore, the stimulation provided by the present invention achieves rapid and sustained improvement over a period of months to attenuate the abnormal portion.

Additionally, the abnormal portion is treated via the three-phase bone scan, and the precise steps involved in which are not described owing to being well known. Refer to FIG. 8B, it illustrates a diagram of the three-phase bone scan before the stimulation according to Example II of the present invention. The drawing contains a shadowed area at in the ankle of left foot, which indicates that the corresponding body flow lines are blocked and result in the abnormal portion.

FIG. 8C diagrammatically illustrates the three-phase bone scan after the stimulation according to Example II of the present invention. In the drawing, the shadow almost disappears after the stimulation. It means that the blockade in the ankle of left foot is resolved, and the abnormal portion is attenuated to recover the normal portion.

Based on the embodiments, the present invention provides an effective and accurate method for determining the stimulated point to attenuate or recover the abnormal portion. Notably, the present invention can be applied in humans, but this does not represent a limitation in its scope. The present invention can also be applied to other various organisms that are equivalent to humans.

According to another aspect of the present invention, the present invention provides a system for determining the stimulated points of the human body, which comprises an input/output module 52, a process system 54, a calculating module 56 and a data base 60, as shown in FIG. 9.

In the system disclosed by the present invention, the input/output module 52, the calculating module 56 and the data base 60 are respectively coupled to the process system 54. The users can input the data, such as the related data of the abnormal portions, to the input/output module 52, and the data are processed by the process system 54. Next, the desired data are selected from the data base 60 thought the calculating module 56, and analyzed and calculated to output the results, such as the data regarding the plurality of the stimulated points and the corresponding stimulation types.

In one embodiment, the process system 54 includes a central processor or a device with the central processor, such as the wire or wireless network device or various communication devices. For example, the network device includes the personal computer (PC), and the communication device includes the cellular phones or the personal digital assistant (PDA).

In one embodiment, the input/output module 52 includes a keyboard or a screen of the network device or the communication device, and the user's data can be inputted by the keyboard or the writeable or touchable screen. The input/output module 52 also includes an infrared ray (IR) sensor. The user's abnormal portions can be sensed by the infrared ray (IR) sensor to read and transmit the data of the abnormal portions. The type of the input/output module 52 for input/output includes the words, the diagram or the three dimensional diagram.

The data base 60 comprises various sub-databases, for example, an abnormal portion sub-database 62, a collateral meridians distribution sub-database 64, a body flow line distribution sub-database 66, a stimulated point sub-database 68. The relationship between the sub-databases is opposite or corresponding. The abnormal portion sub-database 62 includes the data of the abnormal portions, and the abnormal portions include the regions that are sensing certain feelings, including pain. For example, the abnormal region can cover the head, the limbs or trunk or the body, and the visceral organs or nervous system of human body.

In one embodiment, the data base 60 also includes an on-line database, and then the user can access the updating data base through the internet network. In other words, the on-line data base includes the updating abnormal portion, the collateral meridians distribution, the body flow line distribution and the stimulated point sub-databases to obtain the latest data for various users who using the sub-databases.

The calculating module 56 includes various calculating modules. After comparing and analyzing the selected abnormal portion data, the data regarding the plurality of the stimulated point can be calculated and obtained based on the corresponding or (relative) opposite relationships among the theories of the Ying-Yang and the five element shown in FIG. 3, the hexagonal relation shown in FIG. 4 and the seasonal time shown in FIG. 5.

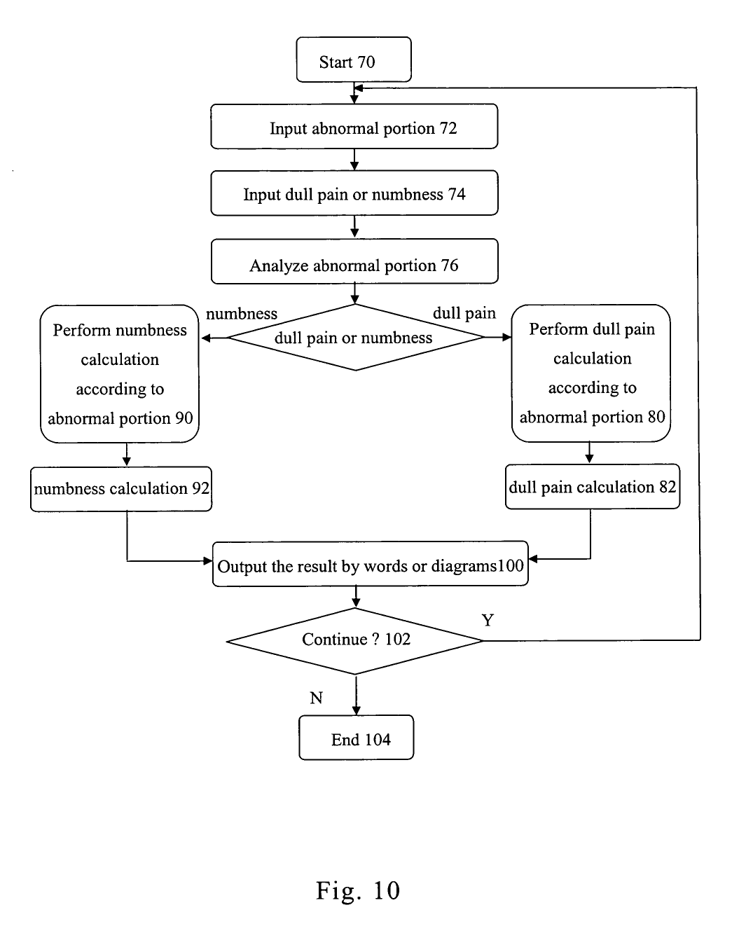

Refer to FIG. 10, it illustrates a flow chart of a system for determining the stimulated point of human body according to the present invention. In step 70, the system is initiated. The user can input the data regarding the abnormal portions in step 72. The abnormal portion data can be read to perform the primary classification. The type of abnormal portion can be identified as dull pain or sharp pain in step 74. The user can analyze the abnormal portion data in step 76, and the abnormal portion can be checked and confirmed by the system in step 78.

When the abnormal portion is identified as dull pain, the system will access the step 80 to perform the dull pain calculation according the analyzed abnormal portion. Based on the inputted abnormal portion data and their corresponding collateral meridians and the body flow lines distributions, the dull pain calculation can be performed in step 82. Then, one or the plurality of the stimulated point can be obtained and determined in view of the corresponding or (relative) opposite relationships among the databases, that is to say, the corresponding or (relative) opposite relationships among the theories of the amended Ying-Yang and the six elements and collateral meridians.

When the abnormal portion is identified as numbness, the system will access the step 90 to perform the numbness calculation according the analyzed abnormal portion. Based on the inputted abnormal portion data and their corresponding seasonal time, the numbness calculation can be performed in step 92. Then, one or the plurality of the stimulated point can be obtained and determined in view of the corresponding seasonal time.

For examples, if the abnormal portion is found on the body flow line AyII, the user can obtain the data that the corresponding seasonal time of the abnormal appearance is spring (wood) and the abnormal appearance usually occurs in autumn, as shown in FIG. 3. According to the data, the user can obtain the stimulated points and the corresponding stimulations performing in the season, the next season, the next to the next season and the last season. Then, the user can offer the appropriate stimulation to attenuate or recover the abnormal portion.

Sequentially, the user can output the calculated and analyzed results by the words or diagrams in step 100. Then, check the system whether to be continued in step 102. In step 104, the system will re-perform the above-mention steps if the remaining data has not processed yet. If not, the system will stop to end the following steps.

According to one aspect of the present invention, the user can input the abnormal portion data by the input/output module 52, process the data by the process system 54, calculate the processed data by the calculating module 56, and then obtain the stimulated point data and the corresponding stimulations. Therefore, the appropriate stimulation can be applied to attenuate or recover the abnormal portion.

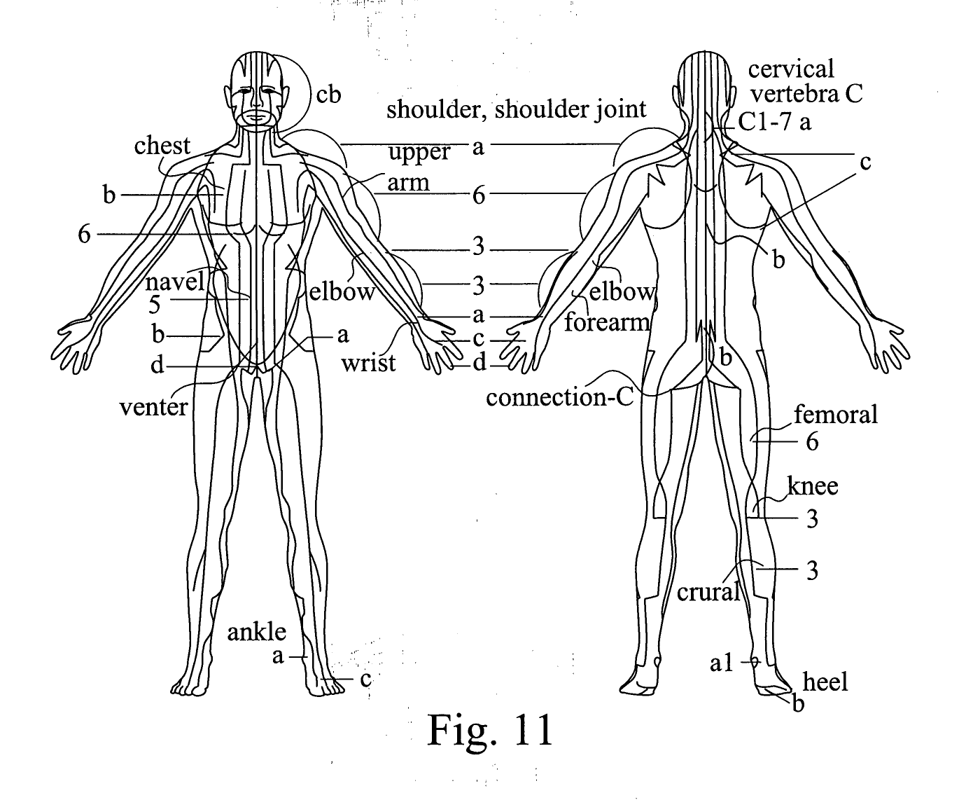

According to another aspect of the present invention, the present invention further provides a module for determining the stimulated points of the human body to offer the distribution data of the plurality of the body flow lines and the stimulated points, as shown in FIG. 11. The module includes the distribution data of the plurality of the body flow lines deposited thereon, and the plurality of the stimulated points are respectively configured on the plurality of the body flow lines, wherein the distribution data of the plurality of the body flow lines and the stimulated points are defined according to the corresponding or (relative) opposite relationships among the theories of the amended Ying-Yang and the six elements and collateral meridians. The abnormal portion can be attenuated or recovered by applying the appropriate stimulations.

The illustrated human body module includes the plurality of the internal body flow lines deposited on the internal portion of the human body modules, and the plurality of the external body flow lines deposited on the external portion of the human body modules. The plurality of the stimulated points is configured respectively on the internal and external body flow lines.

In one embodiment, the internal body flow line can be referred as the Ying body flow line, and the Yang body flow lines can be referred as the external line. The internal body flow lines are configured on the internal portions of hands, feet and internal organs of humans, and the external body flow lines are configured on the external portions of hands, feet and internal organs of humans. For examples, the internal portions of the internal organs include the liver, lung, kidney, heart and spleen. The external portions of the external organs include the stomach, urchin, bladder and large and small intestine.

Refer to the module illustrated in FIG. 11, the user can obtain quickly and determine precisely one or a plurality of stimulated points according to the corresponding and relative relationships between the abnormal portions and their corresponding body flow lines. Then, the corresponding stimulations can be applied to attenuate or recover the abnormal portions. The related descriptions are provided as follows. The following descriptions are used to illustrate the present invention rather than limiting the present invention.

The internal body flow lines are configured on the internal portions of hands and feet, and the external body flow lines are configured on the external portions of the hands and feet. In one embodiment, each internal body flow line of hands includes three body flow lines, TxI, TxII and TxIII, and each external body flow line of hands includes three body flow lines, AxI, AxII and AxIII. Similarly, each internal body flow line of feet includes three body flow lines, TyI, TyII and TyIII, and each external body flow line of feet includes three body flow lines, AyI, AyII and AyIII. Therefore, the human body module includes twelve body flow lines configured thereon. In the present invention, the plurality of the stimulated points and body flow lines are used to illustrate the present invention, but not to limit the same.

According to the above-mentioned aspects, the internal body flow lines TxI, TxII, TxIII of the left hands and the external body flow lines TyI, TyII, TyIII of the right hands are respectively divided into a plurality of stimulated points. The body flow lines are divided into six stimulated points, 1, 2, 3, 4, 5, 6, at the upper elbow of the hand and three stimulated points, a, b, c, below the elbow of the hands. Therefore, each body flow line of the hand can be divided into the nine stimulated points, a, b, c, 1, 2, 3, 4, 5, 6.

The stimulated points configured on TxI are listed as follows. TxI/6 is located at the ventral aspect of the upper arm at the same level as TyI/6, starting from the lateral aspect of the upper arm to the inferior portion of the biceps brachii muscle, and the user can press the superior border of the anterior medial aspect of the humerus. TxI/5 is located superior to the elbow, starting from the medial aspect of the brachialis tendon at the ventral aspect of the upper arm, and the user can press the superior border of the lateral supracondylar ridge of the humerus. TxI/4 is located inferior to the elbow, starting from the medial aspect of the brachioradialis muscle at the ventral aspect of the forearm, and the user can press the depression beside the radial neck. TxI/3 is located at the middle portion of the ventral forearm and at the musculotendinous junction of the brachioradialis muscle, starting from the lateral or medial border of the brachioradialis muscle, and the user can press the depression between the lateral border of the flexor digitorum superficialis muscle and the medial aspect of the radius. Tx I/2 is located at the ventral aspect of the forearm at the same level as TxII/2 (the muscu-lotendinous junction of the flexor pollicis longus muscle), and the user can press the depression between the flexor pollicis longus muscle and the medial aspect of the radius. TxI/1 is located at the ventral aspect of the forearm, posterior aspect of the wrist, starting from the lateral aspect of the flexor carpi radialis tendon, and the user can press the most distal ventral depression of the radius. TxI/a is located at the ventral aspect of the wrist, starting from the lateral border of the flexor carpi radialis muscle, and the user can press the deepest point of the depression formed during the flexion-extension movement of the wrist. TxI/b is located at the ventral aspect of the 1st metacarpal bone, and the user can press the depression beside the junction between the proximal metacarpal base and shaft. TxI/c is located at the ventral aspect of the 1st metacarpal bone, and the user can press the depression beside the junction between the distal metacarpal head and shaft.

The stimulated points configured on TxII are listed as follows. TxII/6 is located at the ventral aspect of the upper arm at the same level as TxI/6, starting from the lateral aspect of the upper arm to the inferior portion of the biceps brachii muscle (or from the medial aspect of the upper arm to the inferior portion of the biceps brachii muscle), and the user can press the posterior border of the anterior medial surface of the humerus. TxII/5 is located superior to the elbow, starting from the lateral border of the biceps brachii tendon at the ventral aspect of the upper arm, and the user can press the most distal central depression of the ventral aspect of the humerus at the inferior portion of the biceps brachii tendon. TxII/4 is located inferior to the elbow, starting from the superior border of the pronator teres muscle at the ventral aspect of the forearm, and the user can press the depression in front of the junction between the proximal radius and ulna. TxII/3 is located at the middle portion of the ventral aspect of the forearm at the same level as TxI/3; starting between the flexor carpi radialis muscle and the palmaris longus muscle, and the user can press the depression between the radius and the ulna. TxII/2 is located at the ventral aspect of the forearm, and the user can draw a curvature along the lateral border of the brachioradialis muscle, and then press the depression at the junction of the curvature, the flexor carpi radialis muscle, and the palmaris longus muscle (TxII meridian). TxII/a is located at the middle portion of the ventral aspect of the wrist, and the user can press the deepest point of the depression formed during the flexion-extension movement of the wrist (not always between the flexor carpi radialis muscle and the palmaris longus muscle). TxII/b is a single point located on the ventral aspect of the palm; press the depression anterior to the junction between the third and fourth metacarpal bases. TxII/c is located at the ventral aspect of the palm; press the depression between the junctions of the second and third distal metacarpal heads and shafts. TxII/c is located at the ventral aspect of the palm, and the user can press the depression between the junctions of the third and fourth distal metacarpal heads and shafts.

The stimulated points configured on TxIII are listed as follows. TxIII/6 is located at the medial aspect of the upper arm at the same level as Tx I/6, starting from the inferior border of the biceps brachii muscle, and the user can press the medial aspect of the humerus. TxIII/5 is located superior to the elbow, starting from the medial aspect of the upper arm, and the user can press the most distal medial-ventral depression of the humerus superior to the medial supracondylar ridge of the humerus. TxIII/4 is located inferior to the elbow, starting from the superior portion of the flexor carpi ulnaris muscle at the medial aspect of the forearm, and the user can press the depression in front of the proximal coronoid process of the ulna. TxIII/3 is located at the middle portion of the ventral forearm at the same level as TxI/3 (musculotendinous junction of the flexor digiti minimi muscle), starting from the superior border of the flexor carpi ulnaris muscle, and the user can press slightly downward horizontally the depression between the flexor digitorum muscles and the ulna. TxII/1 is located at the ventral aspect of the forearm and the posterior portion of the wrist, starting from the lateral aspect of the flexor carpi ulna muscle, and the user can press the ventral depression of the junction between the ulnar head and the shaft at the lateral aspect of the anterior border of the ulna. TxIII/a is located at the ventral aspect of the wrist, starting from the lateral border of the flexor carpi ulnaris muscle, and the user can press the deepest point of the depression formed during the flexion-extension movement of the wrist. TxIII/b is located at the ventral aspect of the palm, and the user can press the medial depression at the hook of the hamate of the wrist. TxIII/c is located at the ventral aspect of the palm, and the user can press the depression between the junctions of the fourth and fifth distal metacarpal heads and shafts.

The stimulated points configured on TyI are listed as follows. Ty I/6 is located at the lateral aspect of the upper arm, and the user can draw a curvature downward along the lateral border of the deltoid muscle to the same level as the junction of the humerus, starting from the anterior border of the brachialis muscle, and press the inferior depression at the deltoid tuberosity of the humerus (lateral surface of the humerus). TyI/5 is located superior to the elbow, starting from the lateral aspect of the upper arm, and the user can press the most distal lateral-ventral depression of the humerus superior to the lateral supracondylar ridge of the humerus. TyI/4 is located inferior to the elbow, starting from the lateral aspect of the forearm and the anterior border of the extensor carpi radialis muscle, and the user can press the depression beside the proximal radial neck. TyI/3 is located at the middle portion of the dorsal forearm at the same level as TxI/3 and the musculotendinous junction of the extensor carpi radialis brevis muscle, starting from the medial border of the extensor carpi radialis brevis muscle, and the user can press the depression between the lateral border of the extensor digitorum muscle and the medial aspect of the radius. TyI/1 is located at the dorsal aspect of the forearm and the posterior portion of the wrist and the user can press the most distal medial depression of the radius. TyI/a is located at the lateral aspect of the dorsal wrist; starting from the extensor carpi longus and extensor carpi brevis muscles, press the deepest point of the depression formed during the flexion-extension movement of the wrist. TyI/b is located at the dorsal aspect of the palm, starting from the second metacarpal bone, and the user can press the ventral depression of the junction between the proximal metacarpal base and shaft. (The pressed points also can be suggested to change “dorsal aspect of the palm” to “dorsal aspect of the hand” or “back of the hand”). TyI/c is located at the dorsal aspect of the palm, starting from the lateral aspect of the second metacarpal bone, and the user can press the ventral depression of the junction between the distal metacarpal head and shaft.

The stimulated points configured on TyII are listed as follows. TyII/6 is located at the lateral aspect of the upper arm at the same level as TyI/6, between the posterior border of the lateral head of the triceps brachii muscle and the long head of the triceps brachii muscle, and the user can press the humerus. TyII/5 is located superior to the elbow, starting from the lateral aspect of the tendon of the long head of the triceps brachii muscle at the posterior upper arm, and the user can press the dorsal depression at the superior portion of the most distal olecranon fossa of the humerus. TyII/4 is located inferior to the elbow, and the user can press the depression in front of the olecranon fossa between the anconeus muscle and the posterior border of the proximal ulna. TyII/3 is located at the middle portion of the dorsal forearm at the same level as TyI/3, starting from the extensor digitorum muscle, and the user can press the depression between the radius and the ulna. TyI/2 is located at the dorsal aspect of the forearm at the same level as TxII/2 (the musculotendinous junction of the extensor pollicis longus muscle); starting from the extensor digitorum muscle, press the depression between the radius and the ulna. TyII/1 is located at the dorsal aspect of the forearm and the posterior portion of the wrist, starting from the lateral aspect of the extensor digitorum tendon, and the user can press the posterior depression at the junction between the distal radius and the distal ulna. TyII/a is located at the middle portion of the dorsal wrist, starting from the lateral border of the extensor digitorum tendon, and the user can press the deepest point of the depression formed during the flexion-extension movement of the wrist. TyII/b is located at the dorsal aspect of the palm, and the user can press the depression anterior to the junction between the third and fourth proximal metacarpal bases. TyII/b is located at the dorsal aspect of the palm, and the user can press the depression anterior to the junction between the fourth and fifth proximal metacarpal bases. TyII/c is located at the dorsal aspect of the palm, and the user can press the depression at the junction between the third and fourth distal metacarpal heads and shafts. TxII/c is located at the dorsal aspect of the palm, and the user can press the depression at the junction between the fourth and fifth distal metacarpal heads and shafts.

The stimulated points configured on TyIII are listed as follows. TyIII/6 is located at the posterior aspect of the upper arm at the same level as TyI/6, starting from the long head and the medial head of the triceps brachii muscle, and the user can press the humerus. TyIII/5 is located superior to the elbow, starting from the medial aspect of the long head of triceps brachii muscle tendon at the posterior portion of the upper arm, and the user can press the most distal dorsal depression of the humerus. TyIII/4 is located inferior to the elbow, starting from the medial aspect of the posterior border of the ulna at the posterior aspect of the forearm, and the user can press the depression anterior to the olecranon of the proximal ulna. TyIII/3 is located at the middle portion of the dorsal forearm at the same level as TyI/3 (the musculotendinous junction of the extensor digiti minimi muscle), starting from the superior border of the extensor carpi ulnaris muscle, and the user can press the depression between the extensor digitorum muscle and the ulna. TyIII/2 is located at the dorsal forearm at the same level as TyII/2, starting from the superior border of the extensor carpi ulnaris muscle, and the user can press the depression between the extensor digitorum muscle and the ulna. TyIII/1 is located at the dorsal aspect of the forearm and the posterior portion of the wrist, and the user can press the lateral depression at the most distal ventral aspect of the ulna. TyIII/a is located at the ulnar aspect of the wrist, starting from the inferior border of the extensor carpi ulnaris muscle, and the user can press the deepest point of the depression formed during the flexion-extension movement of the wrist. TyIII/b is located at the ulnar aspect of the palm, and the user can press the depression beside the junction between the ventral aspect of the fifth proximal metacarpal base and shaft. TyIII/c is located at the ventral aspect of the fifth metacarpal bone, and the user can press the depression beside the junction between the distal metacarpal head and shaft.

According to the above-mentioned aspects, the internal body flow lines AxI, AxII, AxIII of the left feet and the external body flow lines AyI, AyII, AyIII of the right feet are respectively divided into a plurality of stimulated points. In the drawing, the body flow lines are divided into three stimulated points, a, b, c, below the ankle and six stimulated points, 1, 2, 3, 4, 5, 6, above the ankle. Therefore, each body flow line of the foot can be divided into the nine stimulated points, a, b, c, 1, 2, 3, 4, 5, 6.

The stimulated points configured on AxI are listed as follows. AxI/6 is located at the medial aspect of the thigh, starting from the depression between the rectus femoris muscle, the vastus medialis muscle, and the adductor longus muscle, and the user can press the femur. AxI/5 is located superior to the knee joint, starting from the anterior medial aspect of the distal thigh and the middle portion of the vastus medialis muscle, and the user can press the depression at the junction of the medial femoral condyle and femoral shaft. AxI/4 is located inferior to the knee joint, medial aspect of the proximal leg, and the user can press the depression behind the transition point of the medial border of the proximal tibia. AxI/3 is located at the middle portion of the medial aspect of the leg; starting from the level where the medial head of the gastrocnemius muscle and musculoaponeurotic junction connect (musculotendinous junction of the flexor digitorum longus muscle), and the user can press the depression posterior to the medial border of the tibia. AxI/2 is located at the medial aspect of the leg, and the user can draw a curvature along the medial head of the gastrocnemius muscle to the same level as the posterior aspect of the medial tibia (AxI meridian), and press the depression posterior to the medial border of the tibia. AxI/1 is located at the medial aspect of the leg at the same level as the musculotendinous junction of the soleus muscle, starting from the posterior aspect of the medial shinbone, and the user can press the tibia. AxI/a is located at the medial aspect of ankle joint, and the user can press the depression between the anterior inferior border of the medial malleolus and the tibialis posterior muscle. AxI/b is located at the medial aspect of the foot, and the user can press the depression ventral to the junction of the proximal metatarsal base and shaft of the first metatarsal bone. AxI/c is located at the medial aspect of the foot, and the user can press the depression ventral to the junction between the distal metatarsal head and shaft of the first metatarsal bone.

The stimulated points configured on AxII are listed as follows. AxII/6 is located at the medial aspect of the thigh at the same level as AxI/6, starting from the depression between the vastus medialis muscle, the adductor longus muscle, and the adductor magnus muscle, and the user can press the medial surface of the femur. AxII/5 is located superior to the knee joint, medial aspect of the distal femur, and the user can press the depression posterior to the medial femoral epicondyle. AxII/4 is located inferior to the knee joint, medial aspect of the proximal leg, starting from the posterior border of the gracilis muscle, and the user can press the depression posterior to the proximal tibia. AxII/3 is located at the middle portion of the medial leg at the same level as AxI/3, starting from the depression anterior to the soleus muscle, and the user can press the posterior aspect of the tibia. AxII/2 is located at the medial aspect of the leg at the same level as AxI/2 (musculotendinous junction of the tibialis posterior muscle), starting from the depression anterior to the soleus muscle, and the user can press the posterior aspect of the tibia. AxII/a is located at the medial aspect of the ankle joint, starting from the posterior border of the tibialis anterior muscle, and the user can press the deepest point of the depression formed during the flexion-extension movement of the ankle joint. AxII/b is located at the dorsal aspect of the foot, and the user can press the depression anterior to the junction of the proximal metatarsal bases of the first and second metatarsal bones. AxII/c is located at the dorsal aspect of the foot, and the user can press the depression between the junctions of the distal metatarsal heads and shafts of the first and second metatarsal bones.

The stimulated points configured on AxII are listed as follows. AxIII/6 is located at the medial aspect of the thigh at the same level as AxI/6, starting from the depression between the medial adductor longus muscle and the gracilis muscle, and the user can press the femur. AxIII/6 is located at the medial aspect of the thigh at the same level as AxI/6, starting from the depression between the gracilis muscle and the semimembranosus muscle, and the user can press the femur. AxII/5 is located posterior to the knee joint, medial aspect of the distal thigh, starting from the superior border of the gracilis muscle, and the user can press the depression at the junction of the posterior aspect of the medial femoral condyle and the femoral shaft. AxIII/5 is located posterior to the knee joint, posterior aspect of the distal thigh, starting from between the gracilis muscle and the semimembranosus muscle, and the user can press the depression at the junction of the posterior aspect of the medial femoral condyle and the femoral shaft. AxII/4 is located inferior to the knee joint, posterior aspect of the proximal leg, starting from the anterior border of the medial head of the gastrocnemius muscle, and the user can press the posterior depression of the proximal tibia. AxII/3 is located at the middle portion of the medial leg at the same level as AxI/3, starting from the depression between the soleus muscle and the aponeurosis of the gastrocnemius muscle, and the user can press the posterior surface of the tibia. AxIII/2 is located at the medial aspect of the leg at the same level as AxI/2, starting from the depression between the posterior border of the soleus muscle and the aponeurosis of the gastronemius muscle, and the user can press the posterior surface of the tibia. AxIII/a is located at the medial aspect of the ankle joint, posterior to the most superior point of the medial ankle, and the user can press the deepest depression at the posterior border of the medial ankle. AxIII/b is located at the medial aspect of the foot, and the user can press the depression inferior to the junction of the posterior inferior aspect of the tuberosity of navicular and the calcaneus. AxIII/c is located at the sole of the foot, and the user can press the depression between the junctions of the distal metatarsal heads and shafts of the first and second metatarsal bones.

The stimulated points configured on AyI are listed as follows. AyI/6 is located at the anterior lateral aspect of the thigh at the same level as AxI/6 is starting from the depression at the lateral border of the rectus femoris muscle, and the user can press the femur. AyI/5 is located superior to the knee joint, anterior aspect of the distal thigh, starting from the lateral border of the rectus femoris muscle, and the user can press the depression at the junction between the lateral femoral condyle and shaft. AyI/4 is located inferior to the knee joint, lateral aspect of the proximal leg, starting from the posterior border of the tibialis anterior muscle, and the user can press the depression at the junction between the lateral condyle of the tibia and shaft. AyI/3 is located at the middle portion of the lateral aspect of the leg, at the same level as AxI/3 (musculotendinous junction of the extensor digitorum longus muscle), and the user can press the depression at the posterior border of the tibialis anterior muscle. AyI/2 is located at the lateral aspect of the leg, at the same level as AxI/2 (musculotendinous junction of the tibialis anterior muscle), and the user can press the depression at the posterior border of the tibialis anterior muscle. AyI/1 is located at the lateral aspect of the leg, at the same level as AxI/1 (musculotendinous junction of the peroneus tertius muscle), and the user can press the depression at the posterior border of the tibialis anterior muscle. AyI/a is located at the anterior aspect of the ankle joint, starting from between the extensor digitorum longus and extensor digitorum brevis muscles, and the user can press the deepest point of the depression formed during movement of the ankle joint. AyI/b is located at the dorsal aspect of the foot, and the user can press the depression anterior to the junctions of the proximal metatarsal bases and shafts of the second and third metatarsal bones. AyI/c is located at the dorsal aspect of the foot, and the user can press the depression between the junctions of the distal metatarsal heads and shafts of the second and third metatarsal bones.

The stimulated points configured on AyII are listed as follows. AyII/6 is located at the lateral aspect of the thigh, at the same level as Ay I/6, starting from the superior border of the iliotibial tract, and the user can press the lateral surface of the femur. AyII/5 is located superior to the knee joint, lateral surface of the distal thigh, and the user can press the depression posterior to the lateral femoral epicondyle. AyII/4 is located inferior to the knee joint, lateral surface of the proximal leg, starting from the posterior border of the extensor digitorum longus muscle, and the user can press the depression inferior to the proximal fibular head. AyII/3 is located at the middle portion of the lateral aspect of the leg, at the same level as AyI/3, and the user can press the depression at the lateral surface of the fibula. AyII/2 is located at the lateral aspect of the leg and at the same level as AyI/2, and the user can press the depression at the lateral surface of the fibula. AyII/1 is located at the lateral aspect of the leg and at the same level as AyI/1, and the user can press the depression at the anterior surface of the fibula. AyII/a: is located at the lateral aspect of the ankle joint, anterior inferior border of the lateral ankle, and the user can press the deepest depression at the posterior border of the anterior talofibular ligament. AyII/b is located at the dorsal aspect of the foot, and the user can press the depression anterior to the junction of the proximal metatarsal bases of the third and fourth metatarsal bones. AyII/b is located at the dorsal aspect of the foot, and the user can press the depression anterior to the junction of the proximal metatarsal bases of the fourth and fifth metatarsal bones. AyII/c is located at the dorsal aspect of the foot, and the user can press the depression between the junctions of the distal metatarsal heads and shafts of the third and fourth metatarsal bones. AyII/c is located at the dorsal aspect of the foot, and the user can press the depression between the junctions of the distal metatarsal heads and shafts of the fourth and fifth metatarsal bones.

The stimulated points configured on AyIII are listed as follows. AyIII/6 is located at the lateral aspect of the thigh, at the same level as AyI/6, starting from the depression between the iliotibial tract and the biceps femoris muscle, and the user can press the femur. AyIII/6 is located at the posterior aspect of the thigh and at the same level as AyI/6, starting from the depression at the medial border of the biceps femoris muscle, and the user can press the posterior aspect of the femur. AyIII/5 is located at the posterior aspect of the knee, starting from the inferior border of the iliotibial tract, and the user can press the depression at the junction of the posterior aspect of the lateral femoral condyle and femoral shaft. AyIII/5 is located at the posterior aspect of the knee, starting from the depression at the medial border of the biceps femoris muscle, and the user can press the depression at the junction of the posterior aspect of the lateral femoral condyle and femoral shaft. AyIII/4 is located at the posterior aspect of the knee; starting from between the medial and lateral heads of the gastronemius muscle, press the posterior depression of the proximal tibia. AyIII/4 is also located at the posterior aspect of the knee, starting from the lateral border of the lateral head of the gastrocnemius muscle, and the user can press the posterior depression of the proximal tibia. AyIII/3 is located at the middle portion of the posterior aspect of the leg, at the same level as AyI/3. First press the inferior border of the inferior junction between the medial and the lateral heads of the gastrocnemius muscle in order to make the lateral head of the gastrocnemius muscle more evident. Starting from the level of the musculoaponeurotic junction, and the user can press the depression between the soleus muscle and the posterior border of the fibula. AyIII/2 is located at the lateral aspect of the leg and at the same level as AyI/2, and the user can press the depression at the posterior surface of the fibula. AyIII/1 is located at the lateral aspect of the leg and at the same level as AyI/1, and the user can press the depression at the posterior surface of the fibula. AyIII/a is located at the lateral aspect of the ankle joint, posterior to the most superior point of the lateral malleolus, and the user can press the deepest depression at the posterior border of the lateral malleolus. AyIII/b is located at the lateral aspect of the foot, and the user can press the ventral depression at the junction of the proximal metatarsal base and shaft of the fifth metatarsal bone. AyIII/c is located at the lateral aspect of the foot, and the user can press the ventral depression at the junction of the distal metatarsal head and shaft of the fifth metatarsal bone.

In the embodiments mentioned by the present invention, the listed stimulated points are used to illustrate the present invention rather than limiting the same, and can be modified within the spirit and scope of the present invention. The stimulated points can be modified according to the abnormal portions. For examples, TyII/c is located at the dorsal aspect of the palm, and the user can press the depression at the junction between the third and fourth distal metacarpal heads and shafts, or press the depression at the junction between the fourth and fifth distal metacarpal heads and shafts.

Compared with the prior art, the present invention has the following advantages. The present invention provides a method for determining the stimulated point of the human body to replace the method of prior art, combining the theory of the Ying-Yang and the five elements, 12 collateral meridians, the relationships of inter promotion and inter-restrict of Chinese medicine and pathologic physiology of modern medicine. The present invention also can be modified to change the stimulation types and the stimulated points based on the different levels of the abnormal portion. The present invention can avoid connecting directly with the abnormal portion, and can further resolve the blockade of the body flow lines to attenuate the abnormal portion.

One advantage of the present invention is that the present invention can be easily performed, and does not require any special technologies or instruments.

According to the aspects of the present invention, the frequency and duration of the stimulation and stimulated points can be modified and adjusted according the different level of pain conditions to decrease the obstruction in the body flow lines attenuate or recover the abnormal portion.

According to another aspect of the present invention, the illustrated system including various data bases and the calculating module makes the users easily obtain the plurality of stimulated points by analyzing the abnormal portion data through the calculating module. After offering the appropriate stimulation, the abnormal portion can be attenuated or recovered.

As will be understood by a person skilled in the art, the foregoing preferred embodiments of the present invention are illustrative of the present invention, rather than limiting the present invention. Having described the invention in connection with a preferred embodiment, modification will suggest itself to those skilled in the art. Thus, the invention is not to be limited by this embodiment. Rather, the invention is intended to cover various modifications and similar arrangements included within the spirit and scope of the appended claims, the scope of which should be accorded the broadest interpretation so as to encompass all such modifications and similar structures.

What is claimed is:

1. A system for determining stimulated points of human body, comprising:
an input/output module to input/output data;
a process system coupled to said input/output module;
a data base coupled to said process system; and
a calculating module coupled to said process system,
wherein said process system analyzes said data obtained from said input/output module and said data base, and said input/output module can output the corresponding data of one or a plurality of stimulated points of human body.

2. The system in claim 1, wherein said input/output module includes a keyboard, a screen or an infrared ray (IR) sensor.

3. The system in claim 1, wherein the input/output type of said input/output module includes the words, the two or three dimensional diagrams.

4. The system in claim 1, wherein said data includes the data of the abnormal portions.

5. The system in claim 1, wherein said process system includes a central processor, a wire or wireless network device, and said network device includes the personal computer (PC).

6. The system in claim 1, wherein said process system includes various communication devices, and said communication device includes a cellular phones or a personal digital assistant (PDA).

7. The system in claim 1, wherein said data base includes an abnormal portion sub-database, a collateral meridians distribution sub-database, a body flow line distribution sub-database, a stimulated point sub-database or an on-line database.

8. The system in claim 1, wherein said calculating module is performed based on the corresponding or (relative) opposite relationships among the theories of the Ying-Yang and the five element, the hexagonal relation and the seasonal time.

9. The system in claim 1, wherein said corresponding data of one or a plurality of stimulated points of human body include the data of stimulated points and corresponding stimulation types.

10. The system in claim 9, wherein said data of the corresponding stimulation types include acupressure, massage, acupuncture, electric stimulation (ES), laser or the improved chemical or natural articles, said improved chemical or nature articles can be used externally or internally.

11. A module for determining stimulated points of human body, comprising:
a plurality of body flow lines configured on said module; and
a plurality of stimulated points configured respectively on each of said body flow lines,
wherein said plurality of body flow lines and said plurality of stimulated points are defined by the theories of the amended Ying-Yang and the six elements and collateral meridians, and said abnormal portion can be attenuated or recovered after applying stimulations.

12. The module in claim 11, wherein said plurality of body flow lines include the internal and external body flow lines, said internal body flow lines is the Ying body flow lines, and said external body flow lines in the Yang body flow lines.

13. The module in claim 11, wherein distribution of said internal body flow lines include the internal portions of hands, feet and internal organs of humans, and said internal portions of internal organs include the liver, lung, kidney, heart and spleen.

14. The module in claim 11, wherein distribution of said external body flow lines include the external portions of hands, feet and internal organs of humans, and said external portions of external organs include the stomach, urchin, bladder and large and small intestine.

15. The module in claim 11, wherein said types of said abnormal portions include chronic and acute abnormal portions.

16. The module in claim 11, wherein said types of abnormal portions include local and central abnormal portions.

17. The module in claim 11, wherein said abnormal portions include the regions sensing pains, said regions sensing pains include head, limbs, trunk, visceral organs or neural systems.

18. The module in claim 11, wherein said plurality of stimulated points can be the opposite or corresponding portions of said abnormal portions.

19. The module in claim 11, wherein said plurality of stimulated points includes control stimulated points and function stimulated points, said control stimulated points is the stimulated points between said plurality of body flow lines, and said function stimulated points are used for attenuating or recovering said abnormal portions.

20. The module in claim 11, wherein said stimulation types include acupressure, massage, acupuncture, electric stimulation (ES), laser or the improved chemical or natural articles, said improved chemical or nature articles can be used externally or internally.
